(19) United States
(12) Patent Application Publication
(10) Pub. No.: US 2015/0209403 A1
(43) Pub. Date: Jul. 30, 2015

(54) Dose Adjustment

(71) Applicant: AbbVie Inc., North Chicago, IL (US)

(72) Inventors: Walid M. Awni, Green Oaks, IL (US); Prajakta Badri, San Mateo, CA (US); Daniel E. Cohen, Wilmette, IL (US); Sandeep Dutta, Lincolnshire, IL (US); Amit Khatri, Waukegan, IL (US); Rajeev M. Menon, Buffalo Grove, IL (US); Thomas Podsadecki, Northbrook, IL (US); Akshanth Polepally, Waukegan, IL (US); Roger Trinh, Chicago, IL (US); Tianli Wang, Lincolnshire, IL (US); Jiuhong Zha, Chicago, IL (US)

(21) Appl. No.: 14/606,369

(22) Filed: Jan. 27, 2015

Related U.S. Application Data
(60) Provisional application No. 61/932,514, filed on Jan. 28, 2014.
(60) Provisional application No. 62/045,767, filed on Sep. 4, 2014.
(60) Provisional application No. 62/051,066, filed on Sep. 16, 2014.

(51) Int. Cl. A61K 38/06; A61K 38/05; A61K 45/06; A61K 31/513

(52) U.S. Cl. CPC A61K 38/06; A61K 31/513; A61K 38/05; A61K 45/06

(57) ABSTRACT

This application features dose adjustment for drugs co-administered with Compound 1, Compound 2 and/or Compound 3.

### FIELD OF THE TECHNOLOGY

This application relates to dose adjustment for drugs coadministered with Compound 1/r. Compound 2 and/or Compound 3.

### BACKGROUND

Patients with chronic hepatitis C virus (HCV) infection are at risk for developing progressive liver fibrosis, cirrhosis, portal hypertension, hepatocellular carcinoma and decompensated liver disease. HCV can be cured with antiviral therapy, reducing the risk of morbidity and mortality associated with end-stage liver disease.

For approximately a decade, HCV genotype 1-infected patients have been treated with peginterferon/ribavirin dual therapy resulting in sustained virologic response rates (SVR) of approximately 40-50%. However, substantial limitations to efficacy and tolerability remain as many users suffer from side effects, and viral elimination from the body is often incomplete.

### BRIEF DESCRIPTION OF THE DRAWINGS

FIGS. 1 and 2 depict the clinical design for drug-drug interaction studies.

FIG. 3 shows the effect of 3D regimen on Cₘₐₓ, AUC_{τ} and C_{trough} of HIV protease inhibitors.

FIG. 4 depicts the effect of HIV protease inhibitors on Cₘₐₓ, AUC_{τ} and C_{trough} of Compound 1.

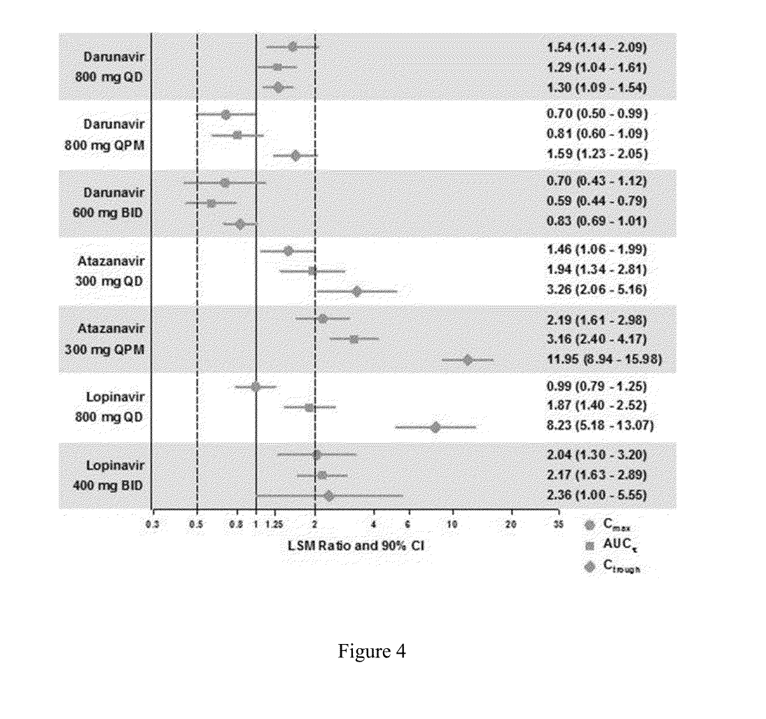

FIG. 5 describes the effect of HIV protease inhibitors on Cmax, AUCτ and Ctrough of ritonavir.

FIG. 6 demonstrates the effect of HIV protease inhibitors on Cₘₐₓ, AUC_{τ} and C_{trough} of Compound 3 (ombitasvir).

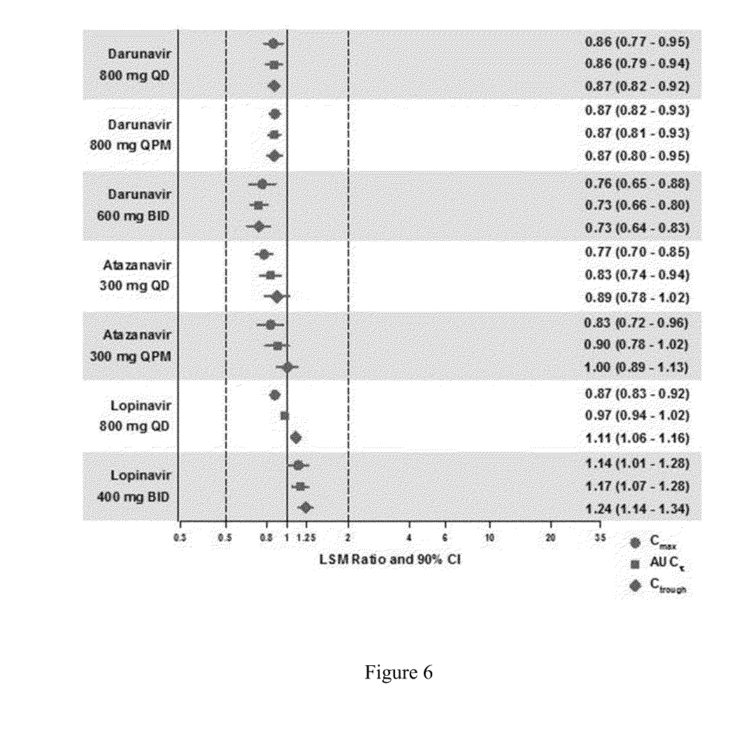

FIG. 7 illustrates the effect of HIV protease inhibitors on Cₘₐₓ, AUC_{τ} and C_{trough} of Compound 2 (dasabuvir).

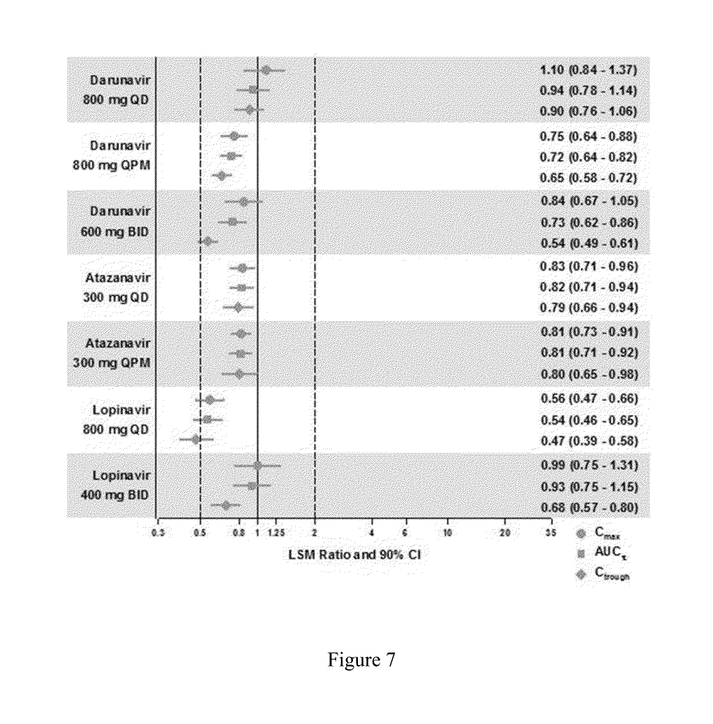

FIG. 8 shows the effect of 3D Regimen on Cₘₐₓ, AUC_{τ} and C_{trough} of HIV-1 ARV drugs.

FIG. 9 depicts the effect of HIV-1 ARV drugs on Cₘₐₓ, AUC_{τ} and Ctrough of Compound 1.

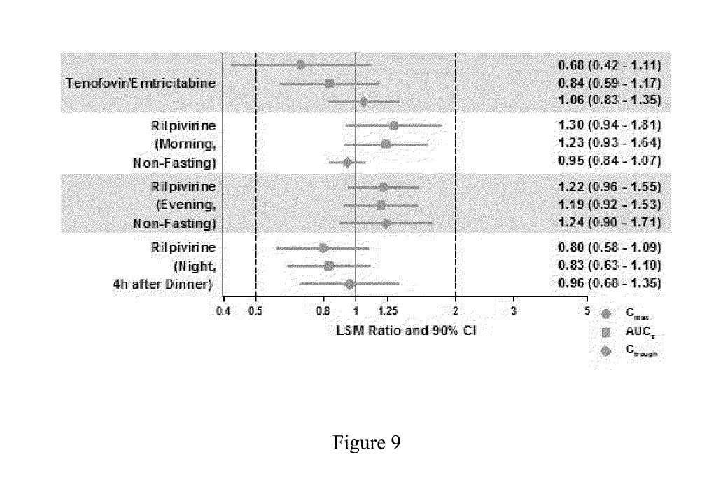

FIG. 10 demonstrates the effect of HIV-1 ARV drugs on Cmax, AUCτ and Ctrough of ritonavir.

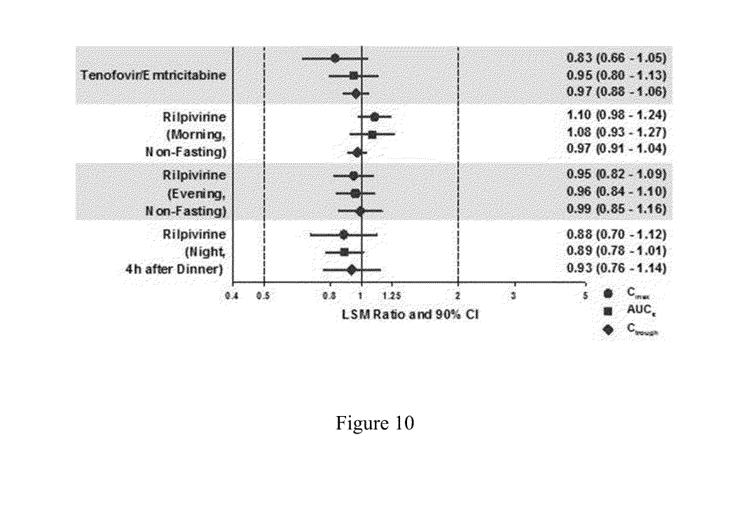

FIG. 11 shows the effect of HIV-1 ARV drugs on Cₘₐₓ, AUC_{τ} and C_{trough} 1 of ombitasvir.

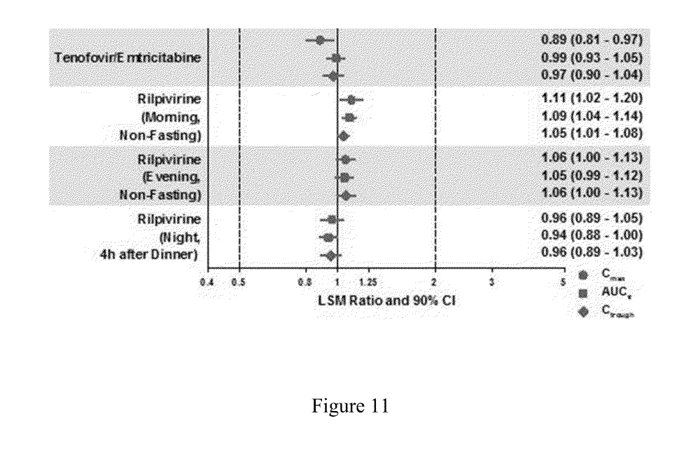

FIG. 12 indicates the effect of HIV-1 ARV drugs on Cₘₐₓ, AUC_{τ} and C_{trough} of dasabuvir.

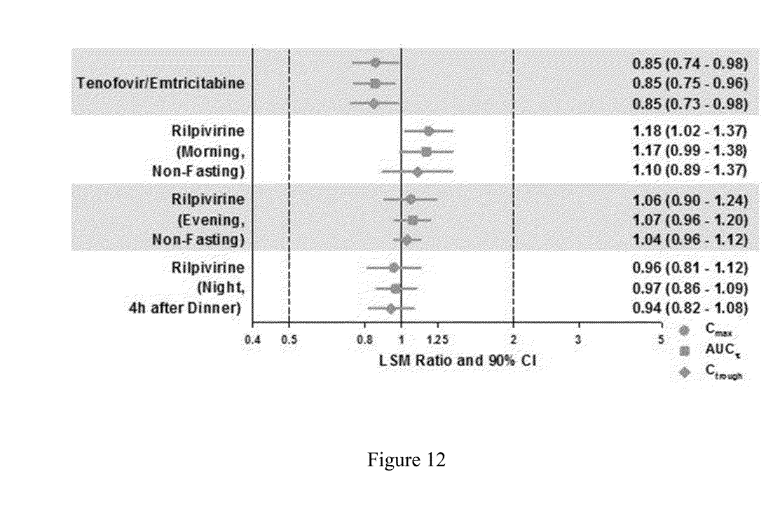

### DETAILED DESCRIPTION

Compound 1, Compound 2 and Compound 3 are potent direct acting agents (DAAs) against HCV. Compound 1

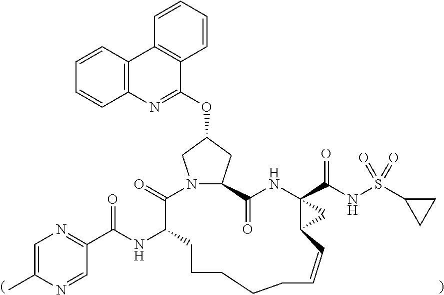 [SMILES: Cc1cnc(C(=O)N[C@H]2CCCCC/C=C\[C@@H]3C[C@@]3(C(=O)NS(=O)(=O)C3CC3)NC(=O)[C@@H]3C[C@@H](Oc4nc5ccccc5c5ccccc45)CN3C2=O)cn1]

is known as (2R,6S,13aS,14aR,16aS,Z)—N-(cyclopropylsulfonyl)-6-(5-methylpyrazine-2-carboxamido)-5,16-dioxo-2-(phenanthridin-6-yloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxamide. The synthesis and formulation of Compound 1 are described in U.S. Patent Application Publication Nos. 2010/0144608 and 2011/0312973, respectively. Compound 1 is typically used with ritonavir. As used herein, “Compound 1/ritonavir” and “Compound 1/r” refer to the combination of Compound 1 and ritonavir, or co-administration of Compound 1 and ritonavir.

Compound 2

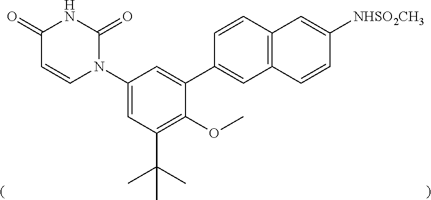

is known as N-(6-(3-tert-butyl-5-(2,4-dioxo-3,4-dihydropyrimidin-1(2H)-yl)-2-methoxyphenyl)naphthalen-2-yl)methanesulfonamide and is described in International Application Publication No. WO2009/039127.

Compound 3

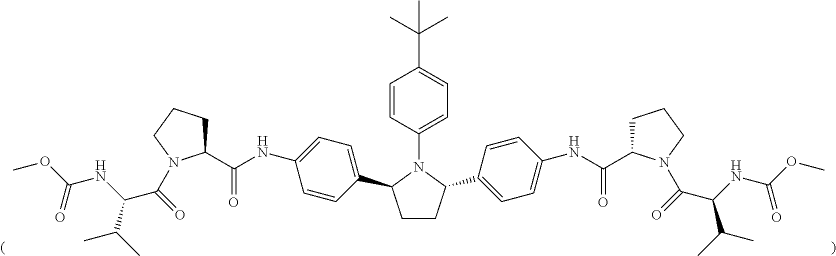

is known as dimethyl(2S,2′S)-1,1′-((2S,2′S)-2,2′-(4,4′-((2S,5S)-1-(4-tert-butylphenyl)pyrrolidine-2,5,diyl)bis(4,1-phenylene))bis(azanediyl)bis(oxomethylene)bis(pyrrolidine-2,1-diyl)bis(3-methyl-1-oxobutane-2,1-diyl)dicarbamate, and is described in U.S. Publication No. 2010/0317568.

The combination of Compound 1/r, Compound 2 and Compound 3, the combination of Compound 1/r and Compound 3, as well as the combination of Compound 1/r and Compound 2, have been shown to be effective against HCV genotype 1. High SVR rates can be achieved when patients infected with HCV genotype 1 are treated with these DAA combos in an interferon-free, short-duration regimen (e.g., a regimen consisting of 8- or 12-week).

HCV patients sometimes have other conditions that may require treatment with other drugs. When Compound 1/r, Compound 2 and/or Compound 3 are used with other drugs, dose adjustment may be needed for the other drugs due to drug-drug interactions. Table 1 summarizes dose adjustment of certain drugs when they are used with Compound 1/r, Compound 2 and Compound 3, or with Compound 1/r and Compound 3. The extent of dose adjustment can be determined for each patient by their physicians or according to the description provided herein.

TABLE 1
Drug-Drug Interaction with Compound
1/r, Compound 2 and/or Compound 3

| Concomitant | |
|---|---|
| Drug | Dose adjustment required? (Y/N) |
| cyclosporine A | Y |
| Tacrolimus | Y |
| Truvada | N |
| (Tenofovir and | |
| Emtricitabine) | |
| Raltegravir | N |
| Darunavir (QD | N |
| and BID) | |
| Darunavir QPM | N |
| Rilpivirine QD | not recommended at approved dose |
| (morning) | |
| Rilpivirine | not recommended at approved dose |
| QPM (evening) | |
| Atazanavir (QD | not recommended at approved dose |
| and QPM) | |
| Digoxin | N** |
| Warfarin | N |
| Methadone | N |
| Kaletra BID | N |
| Buprenorphine/ | N |
| Naloxone | |
| Pravastatin | Y (50% reduction in dose is recommended) |
| Rosuvastatin | Y (limit dose to 10 mg/day) |
| Kaletra QD | Not recommended |
| Omeprazole | N* |
| Oral | N |
| Contraceptives | |
| Carbamazepine | Contraindicated*** |
| Ketoconazole | Y (limit dose to <200 mg/day) |
| Antidepressants | N |
| (Escitalopram | |
| and Duloxetine) | |
| Escitalopram | N |
| Duloxetine | N |
| Gemfibrozil | Contraindicated |
| Efavirenz | Contraindicated |
| Alprazolam | N |
| Zolpidem | N |
| Furosemide | N** |
| Amlodipine | Y (reduce dose by half) |
| Norethindrone | N** |
| Combination | not recommended with the 3D regimen due to |
| oral | potential for increases in ALT |
| contraceptives | but not due to pharmacokinetic interaction |
| containing | |
| ethinyl estradiol | |

*Higher doses may be considered if clinically indicated
**Clinical monitoring recommended
***Contraindicated due to increase in DAA exposures

In one aspect, dose adjustments for the 3D or 2D regimen (see below) are not required when administered with:
CYP3A/P-gP inhibitor, ketoconazole, or other strong inhibitors like itraconazole
CYP2C9 substrate, warfarin, or other substrates (e.g., NSAIDs, oral hypoglycemic agents, sulfonyl ureas)
CYP2C19 substrate, omeprazole and proton pump inhibitors, lansoprazole, esomeraprazole, pantoprazole, etc.
P-gP substrate, digoxin

In another aspect, increase in exposures of OATPIB substrates (pravastatin, rosuvastatin) indicates that lower doses of OATPIB substrates, such as angiotensin II receptor blockers (e.g., valsartan, olmesartan, telmisartan) should be considered when dosed with the 3D regimen.

In another aspect, no dose adjustments are required with the antidepressants, escitalopram or duloxetine (CYP2D6 substrates) and these recommendations can be extrapolated to other antidepressants (e.g., citalopram and fluoxetine).

In another aspect, opioids including methadone (CYP2B6 substrate), buprenorphine or naloxone do not require dose adjustment when dosed with the 3D regimen. Based on results with amlodipine, doses of calcium channel blockers can be reduced by half and monitored when dosed with the 3D regimen.

In another aspect, sleep aids, alprazolam and zolpidem do not require dose adjustments when dosed with the 3D regimen though monitoring is recommended.

In another aspect, ethinyl estradiol contraceptives are not recommended with the 3D regimen. However, the progestin, norethindrone can be dosed with the 3D regimen without dose adjustment.

The 3D regimen does not impact renal cellular transporters (in vitro data) and no interaction is expected with drugs that are cleared renally, e.g., metformin, ACE inhibitors, gabapentin, etc.

Exposures of sensitive CYP3A substrates can be significantly increased by the 3D regimen and exposures of the DAAs can be significantly decreased by CYP3A inducers. Exposures of dasabuvir can be significantly increased by strong CYP2C8 inhibitors. In one aspect, the drugs listed in Table 2 are contraindicated with the 3D regimen.

TABLE 2

| Class | Drug |
|---|---|
| Alpha1-adrenoreceptor | Alfuzosin HCL |
| antagonist | |
| Anticonvulsants | Fusidic acid |
| Antihyperlipidemic | Carbamazepine, phenytoin, |
| | phenobarbital |
| Antimycobacterial | Astemizole, terfenadine |
| Antipsychotic | Blonanserin |
| Benzodiazepines | Oral midazolam, triazolam |
| Ergot derivatives | Ergotamine, dihydroergotamine, |
| | ergonovine, methylergonovin |
| Herbal Product | St. John's Wort |
| HMG-CoA Reductase Inhibitors | Lovastatin, simvastatin |
| Long-acting beta-adrenoceptor | Salmeterol |
| agonist | |
| Neuroleptics | Pimozide |
| Non-nucleoside reverse | Efavirenz |
| transcriptase inhibitor | |
| PDE5 enzyme inhibitor | Sildenafil only when used for the |
| | treatment of pulmonary arterial |
| | hypertension |
| Oral contraceptives | Ethinyl estradiol containing oral |
| | contraceptives |

The majority of commonly used medications evaluated in the DDI studies can be co-administered with the 3D regimen without dose adjustment. In one embodiment, based on results of the drug interaction studies, clinical monitoring with/without dose adjustment is recommended for some concomitant medications. In another embodiment, the strong CYP2C8 inhibitor, gemfibrozil, and sensitive CYP3A substrates and CYP3A inducers are contraindicated with the 3D regimen. In still another embodiment, ethinyl estradiol-containing oral contraceptives are not recommended due to the potential to increase ALT. Progestin only contraceptives, such as norethindrone, can be dosed with the 3D regimen.

According to yet another aspect, the present invention features methods of treating HCV, comprising administering Compound 1/r, Compound 2 and Compound 3, as well as another drug, to a patient in need thereof, wherein the dose of said another drug is adjusted according to Table 1.

In another aspect, the present invention features methods of treating HCV, comprising administering Compound 1/r and Compound 3, as well as another drug, to a patient in need thereof, wherein the dose of said another drug is adjusted according to Table 1.

In any aspect of the invention, the patient preferably is infected with HCV genotype 1.

In any aspect of the invention, the patient preferably is infected with HCV genotype 1a.

In any aspect of the invention, the patient preferably is infected with HCV genotype 1b.

In any aspect of the invention, the patient preferably is a treatment-naïve patient infected with HCV genotype 1.

In any aspect of the invention, the patient preferably is a treatment-naïve patient infected with HCV genotype 1a.

In any aspect of the invention, the patient preferably is a treatment-naïve patient infected with HCV genotype 1b.

In any aspect of the invention, the patient preferably is an interferon null responder infected with HCV genotype 1.

In any aspect of the invention, the patient preferably is an interferon null responder infected with HCV genotype 1a.

In any aspect of the invention, the patient preferably is an interferon null responder infected with HCV genotype 1b.

In any aspect of the invention, the patient preferably is an interferon partial responder infected with HCV genotype 1.

In any aspect of the invention, the patient preferably is an interferon partial responder infected with HCV genotype 1a.

In any aspect of the invention, the patient preferably is an interferon partial responder infected with HCV genotype 1b.

In any aspect of the invention, the patient preferably is an interferon relapser infected with HCV genotype 1.

In any aspect of the invention, the patient preferably is an interferon relapser infected with HCV genotype 1a.

In any aspect of the invention, the patient preferably is an interferon relapser infected with HCV genotype 1b.

In any aspect and preference of the invention, the treatment can, for example, be interferon-free (i.e., does not include administration of interferon) and last for 8 weeks.

In any aspect and preference of the invention, the treatment can, for example, be interferon-free and last for 9 weeks.

In any aspect and preference of the invention, the treatment can, for example, be interferon-free and last for 10 weeks.

In any aspect and preference of the invention, the treatment can, for example, be interferon-free and last for 11 weeks.

In any aspect and preference of the invention, the treatment can, for example, be interferon-free and last for 12 weeks.

In any aspect and preference of the invention, the treatment can, for example, be interferon-free and ribavirin-free, and last for 8 weeks.

In any aspect and preference of the invention, the treatment can, for example, be interferon-free and ribavirin-free, and last for 9 weeks.

In any aspect and preference of the invention, the treatment can, for example, be interferon-free and ribavirin-free, and last for 10 weeks.

In any aspect and preference of the invention, the treatment can, for example, be interferon-free and ribavirin-free, and last for 11 weeks.

In any aspect and preference of the invention, the treatment can, for example, be interferon-free and ribavirin-free, and last for 12 weeks.

In any aspect and preference of the invention, the treatment can, for example, be interferon-free and ribavirin-free, and last for 24 weeks.

In any aspect and preference of the invention, the treatment can, for example, be interferon-free but comprise administration of ribavirin, and last for 8 weeks.

In any aspect and preference of the invention, the treatment can, for example, be interferon-free but comprise administration of ribavirin, and last for 9 weeks.

In any aspect and preference of the invention, the treatment can, for example, be interferon-free but comprise administration of ribavirin, and last for 10 weeks.

In any aspect and preference of the invention, the treatment can, for example, be interferon-free but comprise administration of ribavirin, and last for 11 weeks.

In any aspect and preference of the invention, the treatment can, for example, be interferon-free but comprise administration of ribavirin, and last for 12 weeks.

In any aspect and preference of the invention, the treatment can, for example, be interferon-free but comprise administration of ribavirin, and last for 24 weeks.

The patient treated according to any aspect, example or preference of the invention can, for example, be infected with genotype 2, 3, 4, 5, or 6, instead of genotype 1. The patient treated according to any aspect, example or preference of the invention can, for example, have cirrhosis, or be non-cirrhotic. The patient treated according to any aspect, example or preference of the invention can, for example, be co-infected with HIV and said another drug is an anti-HIV agent. The patient treated according to any aspect, example or preference of the invention can, for example, be a liver transplant recipient.

The doses of Compound 1/r, Compound 2 and Compound 3 typically need not to be adjusted. This applies even in patients with mild, moderate or severe renal impairment or patients with mild or moderate hepatic impairment.

It should be understood that the above description and the following examples are given by way of illustration, not limitation. Various changes and modifications within the scope of the present application will become apparent to those skilled in the art from the present description.

### Example 1

### Cyclosporine A and Tacrolimus

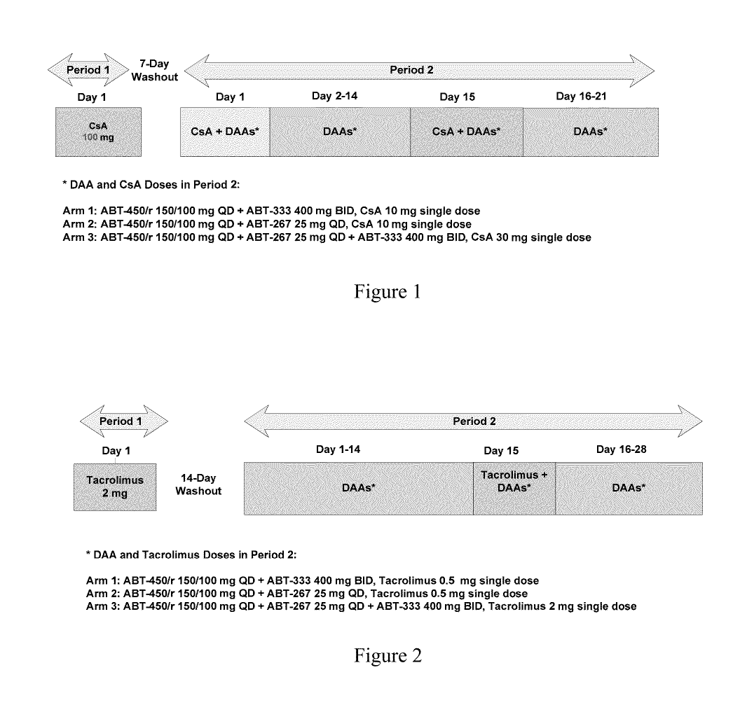

The effect of the DAA combinations (Compound 1/ritonavir and Compound 2; or Compound 1/ritonavir and Compound 3; or Compound 1/ritonavir, Compound 2 and Compound 3) on the pharmacokinetics, safety and tolerability of cyclosporine A (CsA) or tacrolimus was assessed in 72 healthy subjects to provide dosing recommendations in post-liver-transplant subjects with HCV infection. The study designs were shown in FIGS. 1 and 2. Safety was monitored throughout the study.

The magnitude of interaction between the DAA combinations and CsA or tacrolimus was comparable across all arms. CsA+DAAs: On Day 15, CsA dose-normalized (DN) AUC_{inf} and DN_C₂₄ were 4- to 6-fold and 13- to 16-fold, respectively, of CsA exposures when CsA was administered alone. CsA DN_Cₘₐₓ was not affected, while t½ increased from 7 to 24 hours. Compound 1 exposures (Cₘₐₓ and AUC) increased by 12-72% while Compound 2 exposures decreased by 30-34%. Compound 3 and ritonavir exposures were not affected.

Tacrolimus+DAAs: On Day 15, tacrolimus DN_AUC_{inf}, DN_Cₘₐₓ, and DN_C₂₄ were 57- to 86-fold, 3.7- to 4.3-fold, and 17- to 25-fold of tacrolimus exposures when tacrolimus was administered alone, while tacrolimus t½ increased from 29-32 to 232-253 hours. Compound 1, Compound 2 and ritonavir exposures decreased by 11-51% while Compound 3 exposures were not affected. Adverse events were infrequent and mostly mild, and were consistent with those seen with DAAs, CsA or tacrolimus dosed alone.

Concentration-time profiles from these interactions were used to predict dose frequencies that would provide optimal CsA and tacrolimus levels using simulations. When co-dosed with the combination DAA regimens, subjects on CsA should reduce the total daily CsA dose to approximately ⅕^{th} to 1/10^{th} of their previous maintenance dose and/or dosing frequency (e.g., from BID to QD), while subjects on tacrolimus should modify the total daily tacrolimus dose to 0.5 mg/week, to maintain C_{trough} values similar to those prior to DAA co-administration, with appropriate clinical monitoring. Subsequent dose and dosing frequency modifications will be further informed by the individual drug level data.

### Example 2

### Pravastatin and Rosuvastatin

Single doses of Compound 1/r, Compound 2 and Compound 3 (Compound 1/r 150/100 mg QD, Compound 3 25 mg QD, and Compound 2 400 mg BID; hereinafter “3D”), or single doses of Compound 1/r and Compound 3 (Compound 1/r 150/100 mg QD and Compound 3 (ombitasvir) 25 mg QD; hereinafter “2D”), were administered in Period 1. After a washout, multiple doses of pravastatin (10 mg QD, N=12) and rosuvastatin (5 mg QD, N=12) were administered with and without 3D or 2D to steady state in Period 2. DAA doses were: Compound 1/r 150/100 mg QD, Compound 3 (ombitasvir) 25 mg QD and Compound 2 (dasabuvir) 400 mg BID. Intensive PK sampling was performed in both periods and PK parameters estimated by noncompartmental analyses. Safety was evaluated through assessment of adverse events, vital signs, ECG and clinical laboratory tests.

Pravastatin and rosuvastain had no clinically significant effect on 3D or 2D—Cmax and AUC of Compound 1 were <60% higher, ritonavir were ±37% different, ombitasvir and dasabuvir were ±12% different. Pravastatin Cmax and AUC were about 40% and 80% higher, respectively, with 3D or 2D. Rosuvastatin Cmax and AUC were 613% and 159% higher, respectively, with 3D and 161% and 32% higher, respectively, with 2D. No new or unexpected safety findings were observed.

Accordingly, no dose adjustment is necessary for the 3D or 2D regimen during coadministration with pravastatin or rosuvastain. Pravastatin dose should be reduced by 50% and rosuvastatin dose should be no more than 10 mg/day during coadministration with 3D or 2D.

### Example 3

### Anti-Retroviral Agents

Drug-drug interaction (DDI) studies were conducted in healthy volunteers to guide dosing recommendations for several commonly used human immunodeficiency virus-1 (HIV-1) anti-retroviral (ARV) drugs (Table 3) when co-administered with the 3D regimen (i.e., Compound 1/r 150/100 mg QD, Compound 3 25 mg QD, and Compound 2 400 mg BID).

TABLE 3
List of HIV-1 ARV Drugs Evaluated
in DDI Studies with 3D Regimen

| | | HIV-1 ARV dose |
|---|---|---|
| | | and dosing |
| Class of HIV-1 ARV | Name of HIV-1 ARV(s) | Schedule Evaluated |
| Nucleoside/Nucleotide | Emtricitabine (FTC) and | FTC + TDF 200 |
| Reverse Transcriptase | Tenofovir disoproxil | mg + 300 mg QD |
| Inhibitors (N(t)RTIs) | fumarate (TDF) | |
| Non-Nucleoside Reverse | Efavirenz (EFV; | EFV/TDF/FTC |
| Transcriptase Inhibitors | administered as Atripla | 600/300/200 |
| (NNRTIs) | [EFV/TDF/FTC]) | mg QD |
| | Rilpivirine (RPV) | RPV 25 mg QD |
| | | (morning) |
| | | RPV 25 mg QPM |
| | | (evening) |
| | | RPV 25 mg QPM |
| | | (night) |
| Integrase Strand | Raltegravir (RAL) | RAL 400 mg BID |
| Transfer Inhibitors | | |
| (INSTIs) | | |
| Protease Inhibitors (PIs) | Atazanavir (ATV) | ATV 300 mg QD |
| | | (morning) |
| | | ATV 300 mg QPM |
| | | (evening) |
| | Darunavir (DRV) | DRV 800 mg QD |
| | | (morning) |
| | | DRV 800 mg QPM |
| | | (evening) |
| | | DRV 600 mg BID |
| | Lopinavir/ritonavir | LPV/r 400/100 |
| | (LPV/r) | mg BID |
| | | LPV/r 800/200 |
| | | mg QPM |
| | | (evening) |

QPM: QD administration in evening

The pharmacokinetics (PK), safety and tolerability during co-administration of the 3D regimen with the following HIV-1 protease inhibitors at steady state were evaluated:

ATV+ritonavir (RTV) 300+100 mg QD morning or QPM or

DRV+RTV 800+100 mg QD morning or QPM or 600+100 mg BID or

LPV/r 800/200 mg QPM or 400/100 mg BID

Phase 1, single center, randomized, multiple-dose, non-fasting and open-label studies evaluated DDI between the 3D regimen and HIV PIs. Adult male and female subjects in general good health (healthy volunteers) were selected to participate in the DDI studies. All subjects signed an informed consent, approved by an independent Ethics Committee/Institutional Review Board, prior to initiation of any study-related procedures. The 3D Regimen was comprised of Compound 1/r 150/100 mg QD, Compound 3 (ombitasvir) 25 mg QD and Compound 2 (dasabuvir) 400 mg BID in all DDI studies, except for the DDI study of DRV+RTV QD (evening administration) in which dasabuvir 250 mg BID dose using 250 mg optimized tablets was used. Dasabuvir 250 mg optimized tablets provides dasabuvir exposures comparable to the 400 mg tablets used in other studies. Compound 1/r and ombitasvir QD doses were administered in the morning and dasabuvir was administered in morning and evening. Since the 3D regimen contains ritonavir 100 mg, additional doses of RTV for boosting of HIV PIs were not given with morning doses of ATV or DRV during their co-administration with 3D regimen.

Blood samples were collected to quantitate DAAs, ritonavir and HIV PIs in plasma following dosing of the 3D regimen alone (Cohort 1, Study Day 14), HIV PI alone (Cohort 2, Study Day 13 or 14) and 3D regimen+HIV PI co-administration (Cohort 1 and 2, Study Day 27 or 28). Pharmacokinetic (PK) analyses for Compound 1, ombitasvir, dasabuvir, ritonavir and HIV PIs were performed by non-compartmental analysis using Phoenix™ WinNonlin®, Version 6.2 or higher (Pharsight Corporation, St. Louis, Mo.).

Statistical analysis was conducted using SAS, Version 9.2 (Cary, N.C.). Effect of 3D regimen on HIV PI and vice versa was assessed by a repeated measures analysis using log-transformed Cmax, AUCτ (AUC0-24 and AUC0-12 for drugs administered QD and BID, respectively) and Ctrough (C24 and C12 for drugs administered QD and BID, respectively). Least Square Mean (LSM) ratios and 90% confidence intervals (CI) for Cmax, AUCτ and Ctrough were calculated to quantify the magnitude of interaction. Effect of HIV PI on 3D regimen was assessed by utilizing PK data from Study Day 14 and Study Day 28 from Cohort 1. Effect of 3D regimen on HIV PI was assessed by utilizing PK data from Study Day 13 or 14 and Study Day 27 or 28 from Cohort 2.

Safety evaluations performed during the DDI studies included, adverse event (AE) monitoring and vital signs, physical examination, electrocardiogram (ECG), and laboratory tests assessments.

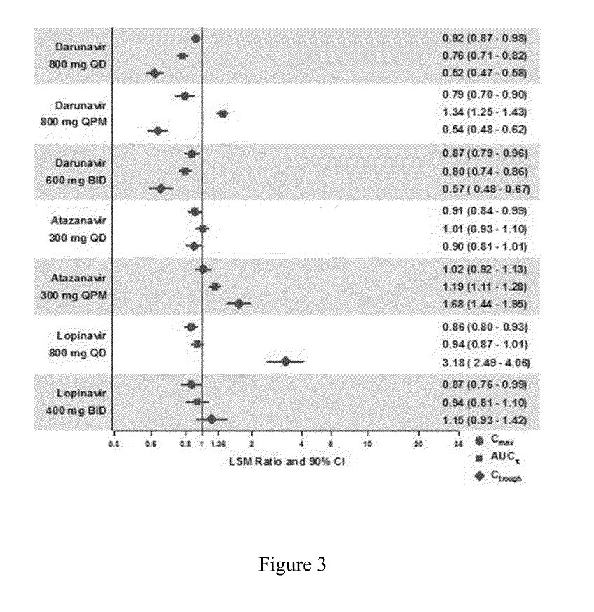

LSM ratios and 90% CI of the Cₘₐₓ, AUC_{τ} and C_{trough} of HIV PI drugs are shown in FIG. 3. LSM ratios and 90% CI of the Cₘₐₓ, AUC_{τ} and C_{trough} of Compound 1, ritonavir, ombitasvir and dasabuvir are shown in Error! Reference source not found.s 4-7. For Compound 1, ritonavir, ombitasvir and dasabuvir, data from Phase 2 studies were used to determine that an increase in exposure by 100% (2×) or a decrease in exposure by 50% (0.5×) did not have a clinically meaningful effect on the safety or efficacy profile of the 3D regimen. For HIV PIs, clinical relevance for magnitude of the interaction was based on data from the prescribing information, regulatory documents or literature.

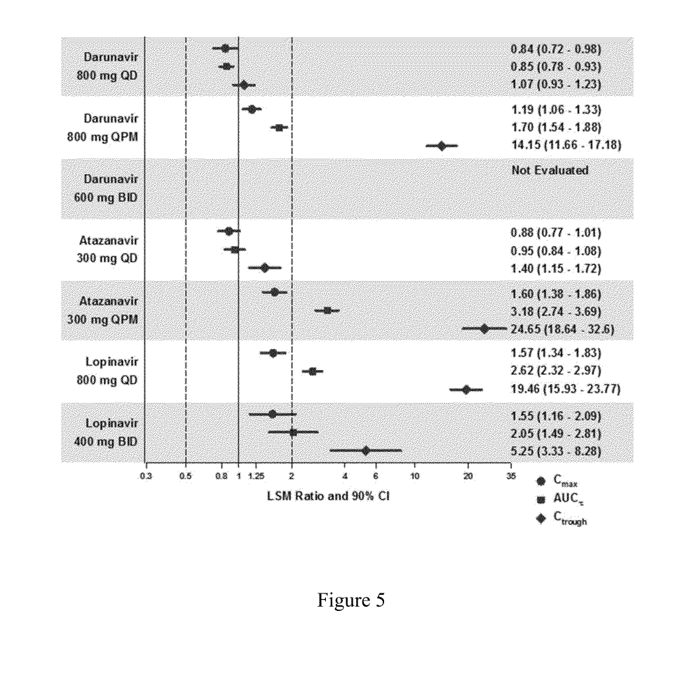

DRV Cₘₐₓ and AUC were minimally affected (up to 24% decrease to 34% increase), but Ctrough were 43% to 48% lower. ATV Cmax, AUC and Ctrough were not affected (≧20% change), except for 68% higher C_{trough} for ATV+RTV QPM. LPV Cₘₐₓ, AUC and C_{trough} were not affected (≦15% change), except for 218% higher LPV Ctrough for LPV/r 800/200 mg QPM. However, LPV C_{trough} during co-administration of LPV/r 800/200 mg QPM with the 3D regimen was comparable to the values observed with LPV/r 400/100 mg BID administered alone. In FIG. 5, ritonavir comparisons for LPV/r BID or QPM regimens is for 300 mg vs. 200 mg; for DRV+RTV QPM and ATV+RTV QPM regimens is for 200 mg vs. 100 mg

During co-administration of 3D regimen with HIV PIs, Compound 1 exposures (Cmax and AUCτ) were minimally to moderately affected (up to approximately ±50% change) with DRV QD, QPM or BID, 46% to 94% higher with ATV+RTV QD and 119% to 216% higher with ATV+RTV QPM, and comparable to 117% higher with LPV/r QPM or BID. The exposures (Cₘₐₓ and AUCτ) of ombitasvir were not affected (≦27% decrease to ≦17% increase) during co-administration of the 3D regimen with HIV PIs. The exposures (Cmax and AUCτ) of dasabuvir were minimally affected (≦46% decrease to ≦10% increase) during co-administration of the 3D regimen with HIV PIs.

The safety and tolerability assessment was based on 14 days of co-administration of 3D regimen with HIV protease inhibitors with data from 12-24 subjects (Cohort 1 and 2 combined) for each DDI assessment. No new safety findings of concern occurred during the DDI studies. No deaths or other serious adverse events were observed. No clinically significant abnormal vital signs, ECG, or laboratory measurements were observed.

During co-administration of 3D regimen with ATV+RTV 300+100 mg QD or QPM, two subjects out of 48, one each on ATV QD and QPM, discontinued the study prematurely due to AE; one subject developed a 1st degree atrioventricular block and one subject developed a macular rash and pruritus. Most common treatment-emergent AE (N≧2): palpitations, ocular icterus (N=5 with ATV+RTV alone and N=3 with 3D+ATV), abdominal pain, infrequent bowel movements, nausea, nasopharyngitis, musculoskeletal chest pain, dizziness, headache, cough, and dermatitis contact. Total bilirubin elevation (Grade 3: 10/24 subjects in ATV QD and 16/24 subjects in ATV QPM), predominantly indirect bilirubin without elevations in aminotransferases, was the most common laboratory abnormality; however, no premature discontinuation was observed due to bilirubin elevations. No subject met Hy's Law criteria. Bilirubin elevations occurred commonly during ATV+RTV dosing alone and did not worsen during co-administration with 3D regimen. No AE of jaundice reported.

During co-administration of 3D regimen with DRV+RTV 800+100 mg QD or QPM or 600+100 mg BID, five subjects out of 60 (1 on DRV QD, 2 on DRV QPM and 2 on DRV BID) discontinued due to rash (N=4) and atrioventricular block first degree (N=1, DRV QPM) that occurred while the subjects were receiving DRV+RTV alone (prior to the addition of the 3D regimen) in Cohort 2. The most common treatment-emergent AE (N≧2): nausea, diarrhea, infrequent bowel movements, dizziness, headache, maculo-papular rash, acne, abnormal dreams and limb discomfort. One subject developed a Grade 2 elevation in alanine aminotransferase (ALT) (without bilirubin elevations) after 10 days of combination dosing with DAAs and DRV that resolved after completing study drug dosing.

During co-administration of 3D regimen with LPV/r 800/200 mg QPM or 400/100 mg BID, no discontinuation due to AE. The most common treatment-emergent AE (N≧2): diarrhea, nausea, nasopharyngitis, dysgeusia, headache, decreased appetite, abdominal pain, abdominal distension, flatulence, fatigue and dysaesthesia. One subject experienced low-grade asymptomatic elevations of ALT (maximum elevation 108 U/L), and 4 subjects experienced total bilirubin elevation (primarily indirect); none resulted in study drug discontinuation and resolved after completing study drug dosing.

Accordingly, co-administration of the 3D regimen with HIV-1 protease inhibitors (ATV, DRV and LPV/r) was generally well tolerated by the subjects. No new safety findings of concern occurred during the DDI studies. No dose adjustment is recommended for the 3D regimen or ATV QD, DRV QD or DRV BID during co-administration. The co-administration of the 3D regimen with LPV/r BID or QPM is not recommended due to a higher dose of ritonavir (300 mg/day) during co-administration, higher incidence of gastrointestinal AEs, and higher Compound 1 exposures.

### Example 4

### Anti-Retroviral Agents

This Example shows the results from DDI studies of HIV-1 N(t)RTIs, NNRTIs and INSTI with 3D regimen. The pharmacokinetics (PK), safety and tolerability during co-administration of the 3D regimen with the following HIV-1 ARV drugs at steady state were evaluated:

FTC 200 mg QD+TDF 300 mg QD (morning administration) or

RAL 400 mg BID or

RPV 25 mg QD (administered in the morning or evening or at night) or

EFV 600 mg QD (administered as EFV/FTC/TDF 600/200/300 mg QD; morning administration)

Phase 1, single center, randomized, multiple-dose, non-fasting and open-label studies evaluated the DDI between the 3D regimen and the HIV-1 ARV drugs. Adult male and female subjects in general good health (healthy volunteers) were selected to participate in the DDI studies. All subjects signed an informed consent approved by an independent Ethics Committee/Institutional Review Board, prior to initiation of any study-related procedures. The 3D regimen is comprised of Compound 1/r 150/100 mg QD, ombitasvir 25 mg QD and dasabuvir 400 mg BID (provides dasabuvir exposure comparable to dasabuvir 250 mg BID dose used in Phase 3 studies using optimized tablet formulation) in all DDI studies included in this presentation. Compound 1/r and ombitasvir QD doses were administered in the morning and dasabuvir was administered in morning and evening.

The morning doses of HIV-1 ARV drugs were administered with morning doses of Compound 1/r+ombitasvir+dasabuvir, and the evening doses of HIV-1 ARV drugs, if applicable, were administered with the evening dose of dasabuvir.

The DDI study of DAAs with EFV was conducted with the 2D regimen of Compound 1/r+dasabuvir.

Blood samples were collected to quantitate DAAs, ritonavir and HIV-1 ARV drugs in plasma following dosing of the 3D (or 2D) regimen alone (Day 14 in Cohort 1), HIV-1 ARV drugs alone (Day 3 for RAL, Day 7 for FTC+TDF and Day 14 for RPV and EFV in Cohort 2) and 3D regimen+HIV-1 ARV co-administration (Day 17 for RAL, Day 21 for FTC+TDF and Day 27 or 28 for RPV DDI study in Cohort 1 and 2). Pharmacokinetic (PK) analyses for Compound 1, ombitasvir, dasabuvir, ritonavir and HIV-1 ARV drugs were performed by non-compartmental analysis using Phoenix™ WinNonlin®, Version 6.2 or higher (Pharsight Corporation, St. Louis, Mo.).

Statistical analysis was conducted using SAS, Version 9.2 (Cary, N.C.). Effect of the 3D regimen on HIV-1 ARV drugs and vice versa was assessed by a repeated measures analysis using log-transformed Cmax, AUCτ (AUC0-24 and AUC0-12 for drugs administered QD and BID, respectively) and Ctrough (C24 and C12 for drugs administered QD and BID, respectively) values at steady state. Least Square Mean (LSM) ratios and 90% confidence intervals (CI) for Cmax, AUCτ and Ctrough were calculated to quantify the magnitude of interaction. Effect of HIV-1 ARV drugs on the 3D regimen was assessed by utilizing PK data from Day 14 and Day 21 (FTC+TDF DDI study) or Day 14 and Day 28 (RPV DDI Study) from Cohort 1. The effect of RAL on DAAs was assessed by cross-study comparison. Effect of the 3D regimen on HIV-1 ARV drugs was assessed by utilizing PK data from Day 7 and Day 21 (FTC+TDF DDI study) or Day 14 and Day 27 or 28 (RPV DDI Study) from Cohort 2 or Day 3 and Day 17 (RAL DDI study).

Safety evaluations performed during the DDI studies included, adverse event (AE) monitoring and vital signs, physical examination, electrocardiogram (ECG), and laboratory tests assessments.

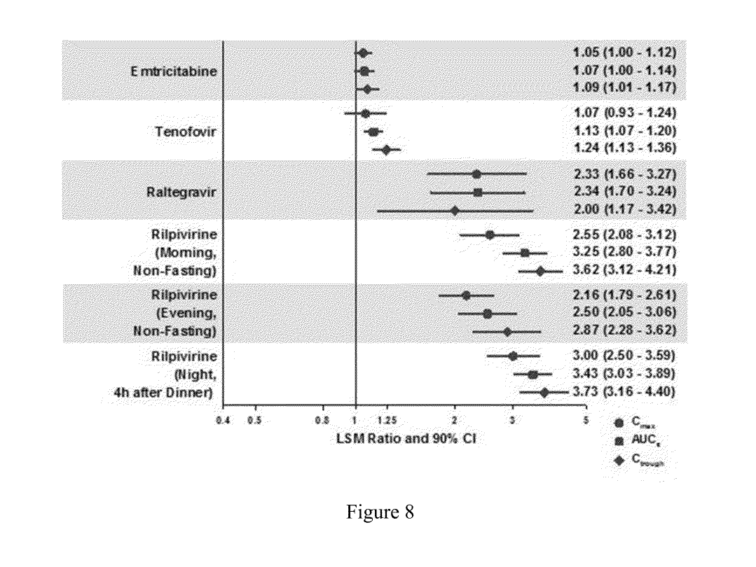

LSM ratios and 90% CI of the Cmax, AUCτ and Ctrough of HIV-1 ARV drugs are shown in FIG. 8. LSM ratios and 90% CI of the Cmax, AUCτ and Ctrough of Compound 1, ritonavir, ombitasvir and dasabuvir are shown in Error! Reference source not found.s 9-12.

For Compound 1, ritonavir, ombitasvir and dasabuvir, data from Phase 2 studies were used to determine that an increase in exposure by 100% (2×) or a decrease in exposure by 50% (0.5×) did not have a clinically meaningful effect on the safety or efficacy profile of the 3D regimen. For HIV ARV drugs, clinical relevance for magnitude of the interaction was based on data from the prescribing information, regulatory documents or literature.

The exposures (Cmax, AUCτ and Ctrough) of FTC and TDF were not affected (5% to 24% increase). The exposures (Cmax, AUCτ and Ctrough) of RAL were 100% to 134% higher. Based on RAL US Prescribing information co-administration of RAL with omeprazole resulted in 212% to 315% increase in RAL Cmax and AUCτ; however, no dose adjustment is recommended due to a lack of safety signals during co-administration of RAL with proton pump inhibitors and H2 blockers in Phase 3 studies of RAL. The exposures (Cmax, AUCτ and Ctrough) of RPV were 116% to 273% higher. Based on RPV US prescribing information, these changes could result in QTc prolongation as a QTcF increase (10.7 msec) was observed at RPV 75 mg QD dose providing RPV Cmax approximately 2.6-fold higher than Cmax observed with RPV 25 mg QD dose. The co-administration of EFV with Compound 1/r+dasabuvir led to premature study discontinuation for several subjects due to adverse events (reviewed below); thus, the PK evaluations could not be performed.

The exposures (Cmax and AUCτ) of Compound 1 and ritonavir were minimally affected (up to ±32% and ±17% change, respectively) during co-administration of 3D regimen with FTC+TDF or RPV, and not affected (within the range of historical data) during co-administration of 3D regimen with RAL. The exposure (Cmax and AUCτ) of ombitasvir were not affected (up to ±11% change) during co-administration of 3D regimen with FTC+TDF or RPV, and not affected (within the range of historical data) during co-administration of 3D regimen with RAL. The exposures (Cmax and AUCτ) of dasabuvir were not affected (up to ±18% change) during co-administration of the 3D regimen with FTC+TDF or RPV, and not affected (within the range of historical data) during co-administration of the 3D regimen with RAL.

The safety and tolerability assessment was based on 14 days of co-administration of 3D regimen with HIV-1 ARV with data from 12-20 subjects (Cohort 1 and 2 combined) for each DDI assessment. No deaths or treatment-emergent serious adverse events (SAE) were observed. During co-administration of 3D regimen with FTC 200 mg QD+TDF 300 mg QD, no treatment discontinuations were observed. The most common treatment-emergent AE (N≧2): headache and abdominal pain. No clinically significant vital signs or laboratory measurements were observed.

During co-administration of 3D regimen with RAL 400 mg BID, no treatment discontinuations were observed. The most common treatment-emergent AE (N=1): dyspepsia, bilirubin (indirect) increase (Grade 2) and alanine aminotransferase (ALT; Grade 1) increase. Bilirubin and ALT elevation occurred in different subjects. No clinically significant vital signs or laboratory measurements were observed.

During co-administration of 3D regimen with RPV 25 mg QD, 2/60 subjects prematurely discontinued from the study due to AE—one each due to blood creatine phosphokinase increase along with aspartate aminotransferase (AST) increase (3D+RPV) and maculopapular rash (RPV alone). One more subject prematurely discontinued from the study based on investigator's discretion (3D+RPV). One subject reported a SAE of spontaneous abortion after completion of the study (25 days following last dose of study drug). The site was unable to contact the subject for further information regarding the reported SAE. The most common treatment-emergent AE (N≧2): constipation, headache, insomnia, oropharyngeal pain, nasopharyngitis, dry skin and dermatitis contact. No clinically significant vital signs or laboratory measurements were observed. No subjects met Hy's Law criteria. Two subjects had an isolated AST/ALT elevation (Grade 2) not accompanied by an elevation in bilirubin that resolved with continued combination dosing.

During co-administration of Compound 1/r+dasabuvir with EFV 600 mg QD (administered as EFV/FTC/TDF 600/200/300 mg QD), nine subjects (Cohort 1: 3 subjects and Cohort 2: 6 subjects) discontinued from the study due to an AE (gastrointestinal AEs; ALT/AST elevations) after receiving Compound 1/r+dasabuvir+EFV/FTCfTDF. Subjects who initiated dosing with EFV/FTC/TDF (Cohort 2) experienced a greater number of AEs (i.e., neurological and gastrointestinal AEs) and elevations in ALT coincident with combination dosing of EFV/FTC/TDF with Compound 1/r+dasabuvir on Study Day 15 compared to those subjects who initiated dosing with Compound 1/r+dasabuvir (Cohort 1) followed by addition of EFV/FTC/TDF on Study Day 15; elevations in ALT were not observed in Cohort 1. The most common treatment-emergent AE (N≧2): headache, dizziness, feeling abnormal, fatigue, blurred vision, nausea, vomiting, increased ALT/AST and hot flush. These safety findings were attributed to EFV, a CYP3A inducer, as the co-administration of 3D regimen with FTC+TDF did not result in such safety events and similar safety findings were observed when CYP3A inducers (rifampin or EFV) were co-administered with ritonavir boosted protease inhibitors (saquinavir/ritonavir, LPV/r) in healthy volunteers.

Accordingly, the co-administration of 3D regimen with FTC+TDF, RAL or RPV was generally well tolerated by the subjects. No new safety findings of concern were identified. No dose adjustment is recommended for the 3D regimen and FTC+TDF or RAL during co-administration. The co-administration of the 3D regimen with RPV resulted in increased RPV exposure (but minimal effect on 3D regimen) and the co-administration of DAAs with EFV resulted in premature study discontinuation due to AEs. Hence, co-administration of 3D regimen with RPV or EFV containing ARV regimens is not recommended.

### Example 5

### Carbamazepine

Single doses of the 3D regimen (Compound 1/r/ombitasvir 150/100/25 mg+dasabuvir 250 mg) were administered alone and in the presence of steady state CBZ 200 mg BID in 12 healthy subjects. Drug concentrations were determined from blood samples collected on multiple study days and PK parameters were estimated by noncompartmental analyses. Safety was assessed (adverse events, vital signs, ECG, etc.) throughout the study.

In the presence of CBZ, Compound 1, ritonavir, ombitasvir and dasabuvir exposures (Cₘₐₓ and AUC) decreased by 66%-71%, 83%-88%, 30%-32% and 55%-70%, respectively. CBZ exposures were minimally affected (≦17% increase). Coadministration of the 3D with CBZ was generally tolerated by the subjects. More treatment-emergent adverse events of nausea and vomiting considered related to CBZ were reported compared to 3D alone. Increases in ALT and AST were observed when 3D was coadministered with CBZ, peaking at Grade 3 in 1 subject.

Accordingly, significant decreases were observed in Compound 1, ritonavir and dasabuvir exposures in the presence of a strong CYP3A4 inducer, CBZ, which could lead to decreases in the therapeutic activity of the 3D regimen. Thus, coadministration of the 3D regimen with strong enzyme inducers such as carbamazepine is not recommended.

### Example 6

### Omeprazole

In 24 healthy volunteers omeprazole (40 mg) were administered on day 1 and days 20-24. The 3D or 2D regimen (Compound 1/r 150/100 mg QD+ombitasvir 25 mg QD±dasabuvir 250 mg BID) was administered on days 6-24. Intensive PK sampling was performed for study drugs when dosed alone and during coadministration and parameters estimated by noncompartmental analyses. Safety was evaluated through assessment of adverse events, vital signs, ECG and clinical laboratory tests.

Omeprazole coadministration did not affect (<20% change) the Cmax, AUC or Ctrough of Compound 1, ritonavir, ombitasvir or dasabuvir from the 3D and the 2D regimens. Coadministration of omeprazole with the 3D or 2D regimen resulted in ˜40 to 50% decrease in omeprazole exposure probably due to mild CYP2C19 induction by ritonavir. No new or unexpected safety findings were observed.

Therefore, no dose adjustment is necessary for the 3D or 2D regimen during coadministration with acid reducing agents. The decrease in omeprazole exposure when coadministered with the 3D or 2D regimen is not expected to have a clinically meaningful impact on omeprazole efficacy. Dose adjustment for omeprazole may not be necessary but an increased dose may be considered based on response.

### Example 7

### Digoxin

Single doses of digoxin (0.5 mg) were administered alone and in presence of steady state Compound 1/r 150/100 mg QD+ombitasvir 25 mg QD+/−dasabuvir 400 mg BID in 24 healthy volunteers. Drug concentrations were

determined from intensive blood samples collected on multiple study days and PK parameters were estimated by noncompartmental analyses. Safety was assessed (adverse events, vital signs, ECG, etc.) throughout the study.

Following coadministration with the 3D or 2D regimens, digoxin exposures (Cmax and AUCinf) increased by 15-16% (3D) and 24-58% (2D). DAA exposures were not affected (≦15% change). No new or unexpected safety findings were observed.

Digoxin exposures increase slightly (≦16%) with 3D while clinically significant increases (≦58%) were observed with 2D. Since digoxin is a narrow therapeutic index (NTI) drug, subjects receiving this drug in combination with 2D or 3D should be monitored, with 30-50% dose decrease recommended with 2D. Since 3D does not cause meaningful P-gp inhibition, P-gp substrates do not require dose adjustment with 3D.

### Example 8

### Ketoconazole

Single doses of Compound 1/r/ombitasvir 150/100/25 mg+/−dasabuvir 250 mg were administered alone and in the presence of steady state KTZ 400 mg QD in 24 healthy volunteers. Drug concentrations were determined from intensive blood samples and PK parameters were estimated by noncompartmental analyses. Safety assessment (adverse events, clinical labs, vital signs, ECG) was done throughout the study.

KTZ increased the Compound 1 Cmax and AUCinf by 37-72% and 98-116%. Exposures of ritonavir, ombitasvir, and dasabuvir were increased by 27-57%, up to 26%, and up to 42%, respectively. Following coadministration with a single dose of 3D or 2D, KTZ Cmax was comparable (<20% change) while AUC24 increased by 105-117% compared to administration alone, due to decrease in KTZ clearance. In this study with 12 subjects/group, the regimens were well tolerated over the 1-day coadministration.

Following coadministration with 3D or 2D, KTZ doses should be limited to less than 200 mg per day. Changes in exposures of Compound 1, ombitasvir, dasabuvir, and ritonavir are not considered clinically significant and no dose adjustments are recommended when the 3D or 2D regimen is coadministered with CYP3A4 inhibitors or Pgp inhibitors.

What is claimed is:

1. A method of treating HCV, comprising administering Compound 1/r, Compound 2 and Compound 3, as well as another drug, to a patient in need thereof, wherein the dose of said another drug is adjusted according to Table 1.

2. A method of treating HCV, comprising administering Compound 1/r and Compound 3, as well as another drug, to a patient in need thereof, wherein the dose of said another drug is adjusted according to Table 1.
